(19) United States
(12) Patent Application Publication
(10) Pub. No.: US 2020/0054305 A1
(43) Pub. Date: Feb. 20, 2020

(54) ULTRASONIC ENDOSCOPE ACOUSTIC LENS AND ULTRASONIC ENDOSCOPE

(71) Applicant: OLYMPUS CORPORATION, Tokyo (JP)

(72) Inventors: Rieko NIINO, Tokyo (JP); Koji KOBAYASHI, Tokyo (JP)

(73) Assignee: OLYMPUS CORPORATION, Tokyo (JP)

(21) Appl. No.: 16/663,111

(22) Filed: Oct. 24, 2019

Related U.S. Application Data
(63) Continuation of application No. PCT/JP2018/010832, filed on Mar. 19, 2018.

(30) Foreign Application Priority Data
May 16, 2017 (JP) 2017-097312

(51) Int. Cl. A61B 8/00; A61B 8/12

(52) U.S. Cl. CPC A61B 8/445; A61B 8/12

(57) ABSTRACT

An ultrasonic endoscope acoustic lens includes a base material, a filler, and a friction reducing agent. The base material is composed of at least one elastomer. The filler is added to the base material. The friction reducing agent is disposed to cover at least a part of a base material surface of the base material and exposed to a lens surface.

### BACKGROUND OF THE INVENTION

### Field of the Invention

The present invention relates to an ultrasonic endoscope acoustic lens and an ultrasonic endoscope.

The application is a continuation application based on a PCT Patent Application No. PCT/JP2018/010832, filed Mar. 19, 2018, whose priority is claimed on Japanese Patent Application No. 2017-097312, filed May 16, 2017. The content of both the PCT Application and the Japanese Application are incorporated herein by reference.

### Description of Related Art

Ultrasonic endoscopes are known as medical endoscopes.

An ultrasonic endoscope is equipped with an ultrasonic transducer for acquiring an image of a subject. An acoustic lens for converging ultrasonic waves is disposed on a surface of the ultrasonic transducer.

The acoustic lens is required to have acoustic characteristics close to acoustic characteristics of biological tissue to efficiently introduce ultrasonic waves into a subject such as biological tissue. For example, when an acoustic impedance of the acoustic lens is close to an acoustic impedance of the biological tissue, surface reflection is reduced at a portion in contact with the biological tissue, and thus the ultrasonic waves are efficiently propagated to the biological tissue.

For example, an acoustic lens that contains an acoustic wave probe silicone resin in which an inorganic compound is added to polysiloxane having a vinyl group for the purpose of improving sensitivity is described in Japanese Unexamined Patent Application, First Publication No. 2016-107076. The inorganic compound contained in the acoustic lens is composed of a material selected from the group consisting of calcium carbonate, aluminum nitride, calcium oxide, vanadium oxide, silicon nitride, barium carbonate, titanium carbide, titanium nitride, copper oxide, zirconium carbide, and tungsten carbide.

### SUMMARY OF THE INVENTION

An ultrasonic endoscope acoustic lens of a first aspect of the present invention includes: a base material composed of at least one elastomer; a filler added to the base material; and a friction reducing agent disposed to cover at least a part of a surface of the base material and exposed to a lens surface.

An ultrasonic endoscope of a second aspect of the present invention includes the ultrasonic endoscope acoustic lens according to the first aspect.

### BRIEF DESCRIPTION OF THE DRAWINGS

FIG. 1 is a schematic front view showing a rough constitution of an ultrasonic endoscope according to a first embodiment of the present invention.

FIG. 2 is a schematic sectional view showing a constitution of main parts of the ultrasonic endoscope according to the first embodiment of the present invention.

FIG. 3 is a schematic sectional view showing an example of an ultrasonic endoscope acoustic lens according to the first embodiment of the present invention.

FIG. 4 is a schematic sectional view showing an example of an ultrasonic endoscope acoustic lens according to a second embodiment of the present invention.

FIG. 5 is a schematic sectional view showing an example of an ultrasonic endoscope acoustic lens according to a third embodiment of the present invention.

FIG. 6 is a schematic sectional view showing an example of an ultrasonic endoscope acoustic lens according to a fourth embodiment of the present invention.

### DETAILED DESCRIPTION OF THE INVENTION

Hereinafter, embodiments of the present invention will be described with reference to the drawings. In all the drawings, even in a case where the embodiments are different, identical or equivalent members are given the same reference signs, and common descriptions are omitted.

### First Embodiment

Hereinafter, an ultrasonic endoscope acoustic lens and an ultrasonic endoscope according to a first embodiment of the present invention will be described.

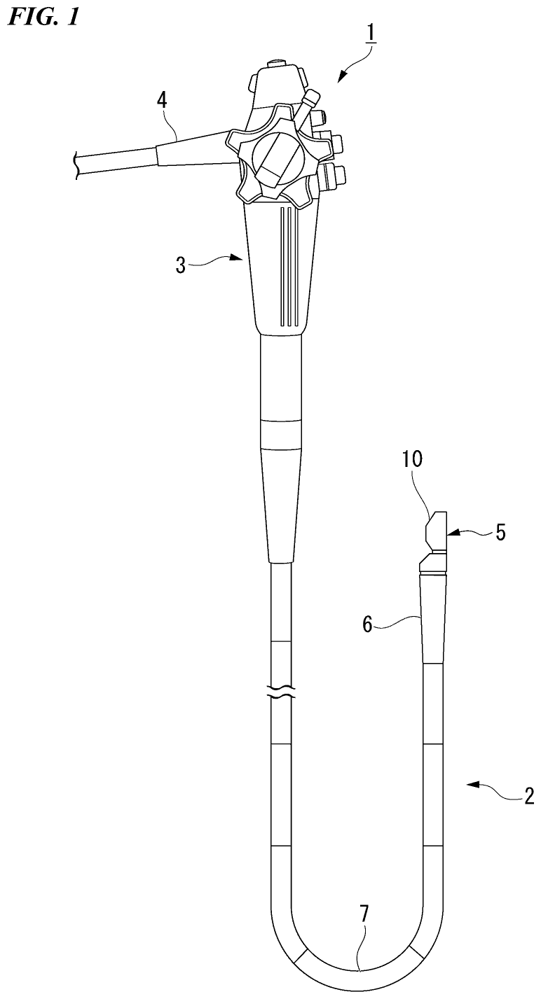

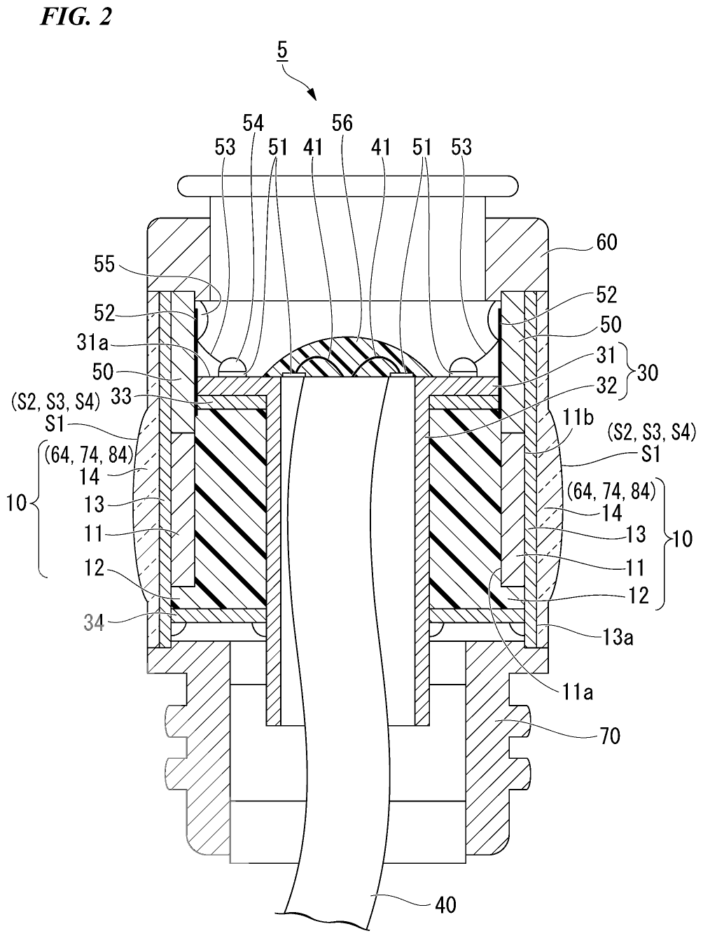

FIG. 1 is a schematic front view showing a rough constitution of an ultrasonic endoscope according to a first embodiment of the present invention. FIG. 2 is a schematic sectional view showing a constitution of main parts of the ultrasonic endoscope according to the first embodiment of the present invention.

As shown in FIG. 1, an ultrasonic endoscope 1 of the present embodiment (an ultrasonic endoscope device) includes an elongate insertion portion 2 to be inserted into a body of a subject, a manipulation portion 3 connected to a proximal end of the insertion portion 2, and a universal cord 4 extending from the manipulation portion 3.

The insertion portion 2 has a configuration in which a rigid distal end portion 5, a bending portion 6, and a flexible tube portion 7 that is thin and long and has flexibility are connected in this order from the distal end thereof. An endoscopic channel through which an endoscopic device is inserted may be provided in the flexible tube portion 7.

As shown in FIG. 2, the rigid distal end portion 5 includes a cylindrical member 30 and a plurality of ultrasonic transducers 10. While not shown separately, in the case where the endoscopic channel is provided in the flexible tube portion 7, an opening used as an exit of the endoscopic channel is formed in the rigid distal end portion 5.

The cylindrical member 30 includes an annular flange 31 and a cylindrical portion 32 that extends from a central edge of the flange 31 in a direction of the flexible tube portion 7 (not shown) (a direction from top to bottom in FIG. 2).

A coaxial cable 40 is inserted into the cylindrical portion 32 of the cylindrical member 30.

Each ultrasonic transducer 10 is a device portion that emits ultrasonic waves to a subject. The plurality of ultrasonic transducers 10 are arranged along an outer circumferential surface of the cylindrical member 30 in a circumferential direction.

Each ultrasonic transducer 10 includes a piezoelectric element 11, a backing material 12, an acoustic matching layer 13, an acoustic lens (an ultrasonic endoscope acoustic lens) 14, and an electrode (not shown).

The piezoelectric element 11 generates ultrasonic vibration when a voltage is applied by the electrode (not shown). The piezoelectric element 11 in the present embodiment is formed in a flat plate shape. One plate surface 11a of the piezoelectric element 11 is disposed at a position at which it faces the cylindrical portion 32 in a radial direction of the cylindrical member 30.

The backing material 12 is a member for absorbing, among ultrasonic vibrations generated by the piezoelectric element 11, the ultrasonic vibrations advancing radially towards the inside of the rigid distal end portion 5 from the plate surface 11a. The backing material 12 is filled between the cylindrical portion 32 and the piezoelectric element 11.

A resin material having a suitable vibration absorption characteristic is used as a material of the backing material 12.

The backing material 12 is sandwiched between annular members 33 and 34 into which the cylindrical portion 32 is inserted in an axial direction of the rigid distal end portion 5.

The annular member 33 is adjacent to the flange 31 and is provided to be in contact with a substrate 50 that extends from the piezoelectric element 11 in a direction of a distal end of the rigid distal end portion 5.

The annular member 34 is provided to be in contact with the acoustic matching layer 13 (to be described below) at a position closer to the flexible tube portion 7 (not shown) than the piezoelectric element 11.

The acoustic matching layer 13 is a layered part that reduces a difference in acoustic impedance between the subject and the piezoelectric element 11. An acoustic impedance of the acoustic matching layer 13 is suitably set according to the acoustic impedance of the subject, and thus reflection of ultrasonic waves at the subject is reduced.

The acoustic matching layer 13 is provided to cover the plate surface 11a and a plate surface 11b opposite to the plate surface 11a in at least the piezoelectric element 11. For this configuration, ultrasonic waves emitted from the plate surface 11b to the outside of the rigid distal end portion 5 in a radial direction via the acoustic matching layer 13 are efficiently introduced into the subject.

The acoustic matching layer 13 may be composed in a single layer or in multiple layers.

The acoustic lens 14 converges ultrasonic waves that are generated by the piezoelectric element 11 and are propagating to the outside of the rigid distal end portion 5 in the radial direction through the acoustic matching layer 13, and emits the converged ultrasonic waves to the outside. The acoustic lens 14 is formed in a suitable shape for converging the ultrasonic waves. For example, a lens surface S1 of the acoustic lens 14 is a curved surface that swells outward. The acoustic lens 14 is provided to cover the acoustic matching layer 13 from the outside of the rigid distal end portion 5 in the radial direction.

A constitution of the acoustic lens 14 will be described in detail after description of the ultrasonic endoscope 1.

In the flange 31 of the cylindrical member 30, a plurality of electrode pads 51 are provided on a surface 31a in a direction opposite to the annular member 33.

Wirings 41 extending from the coaxial cable 40 are connected to the electrode pads 51. The electrode pads 51 and an electrode layer 52 provided on the substrate 50 are connected by wires 53.

The electrode pads 51 and the wires 53 are joined by solder 54. The electrode layer 52 and the wires 53 are joined by solder 55.

To prevent the wirings 41 from being disconnected from the electrode pads 51, for example, due to a load applied to the coaxial cable 40, connection portions between the electrode pads 51 and the wirings 41 are covered with a potting resin 56.

A distal end structural member 60, which covers the connection portions between the electrode pads 51 and the wirings 41, is provided at the distal end of the rigid distal end portion 5. The rigid distal end portion 5 is connected to the bending portion 6 via a connecting member 70.

Next, a detailed constitution of the acoustic lens 14 will be described.

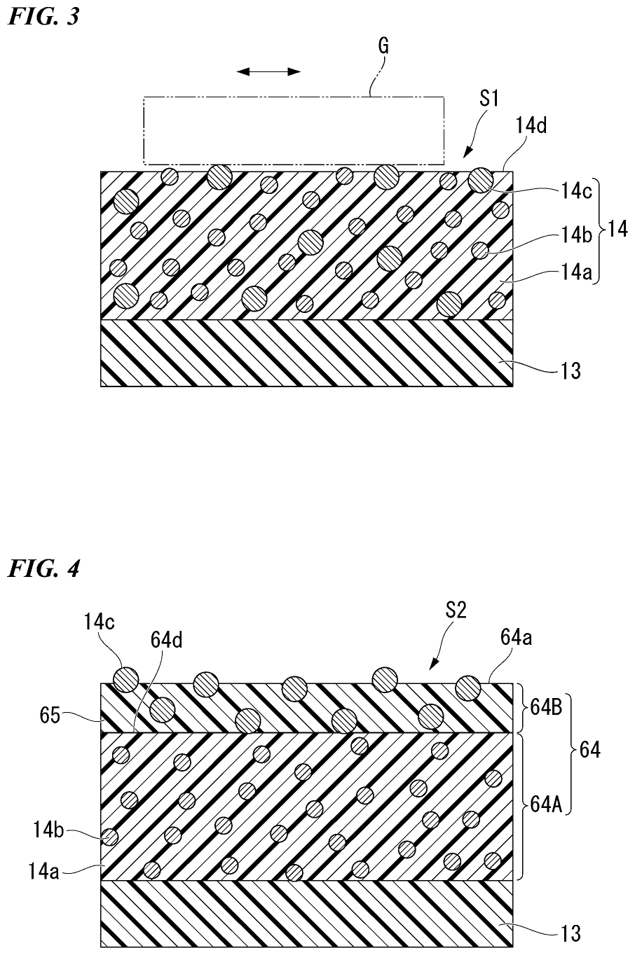

FIG. 3 is a schematic sectional view showing an example of the ultrasonic endoscope acoustic lens according to the first embodiment of the present invention.

As shown in FIG. 3, the acoustic lens 14 according to the present embodiment includes a base material 14a, a filler 14b, and a friction reducing agent 14c.

At least one elastomer having acoustic characteristics close to that of biological tissue of the subject or the like is used for the base material 14a. For example, a diorganopolysiloxane (elastomer) or a silicone rubber compound (elastomer) containing the diorganopolysiloxane as a main agent (which may hereinafter be referred to collectively as “silicone elastomer”) may be used as the base material 14a. However, in the silicone elastomer, a material that is excellent in moldability, adhesiveness, etc. is more preferably. For example, in view of moldability, the silicone is more preferably a non-millable type instead of a millable type. The coefficient of friction of the silicone elastomer that is excellent in moldability, adhesiveness, etc. is likely to be high.

A configuration of the diorganopolysiloxane or the silicone rubber compound containing the diorganopolysiloxane as the main agent, which is used in the base material 14a, is not particularly limited. As a configuration of an organic group in the diorganopolysiloxane and the configuration of the silicone rubber compound containing the diorganopolysiloxane as the main agent, all the configurations described, for example, in Japanese Unexamined Patent Application, First Publication No. S62-11897 can be used.

An adequate amount of the filler 14b is added to the base material 14a. An inorganic filler from which acoustic characteristics required of the acoustic lens 14 are obtained is used for the filler 14b. Here, examples of the acoustic characteristics required of the acoustic lens 14 include, for example, an acoustic impedance, an attenuation factor of ultrasonic waves, and so on.

It is more preferable that a material of the filler 14b have a high density. In this case, even if an added amount of the filler 14b to the base material 14a is small, the acoustic lens 14 can obtain the required acoustic impedance, and thus the attenuation factor of the ultrasonic waves can be reduced.

It is more preferable that the material of the filler 14b have high mechanical strength. In this case, mechanical strength of the acoustic lens 14 is improved. Since a high-density material generally has high mechanical strength, when the density of the filler 14b is high, the mechanical strength of the acoustic lens 14 is easily increased.

Examples of the inorganic filler preferred as the filler 14b include silica, alumina, boehmite, cerium oxide, boron nitride, aluminum nitride, magnesium oxide, aluminum hydroxide, zinc oxide, tungsten trioxide, zirconia, diamond, silicon nitride, silicon carbide, sapphire, and so on. Since any of the inorganic fillers given above by way of example has high mechanical strength, shear strength thereof is also high.

The filler 14b is not limited to one type. A plurality of types of inorganic fillers may be used as the filler 14b.

Since FIG. 3 is a schematic view, the filler 14b is represented in a spherical shape. However, the shape of the filler 14b is not limited to the spherical shape. For example, the shape of the filler 14b may be a granular shape, a polyhedral shape, a plate shape, a rod shape, a fabric shape, an indefinite shape, or the like in addition to the spherical shape.

The amount of the filler 14b in the acoustic lens 14 can be an adequate content by which the acoustic characteristics and the mechanical strength required for the acoustic lens 14 are obtained.

For example, the amount of the filler 14b in the acoustic lens 14 may be 1 part by mass or more and 100 parts by mass or less with respect to 100 parts by mass of the base material 14a.

When there is less than 1 part by mass of the filler 14b, since the added amount of the filler 14b is too small, it may be difficult to adequately adjust the acoustic impedance of the acoustic lens 14, or the mechanical strength of the acoustic lens 14 may not be able to be improved much.

When the filler 14b exceeds 100 parts by mass, the moldability of the acoustic lens 14 is deteriorated, and thus a shape of a molding die may not be accurately transferred. In this case, lens performance of the acoustic lens 14 may be reduced. Furthermore, when the added amount of the filler 14b is increased, the attenuation factor of ultrasonic waves may be increased.

The friction reducing agent 14c is exposed to at least a part of the lens surface S1 of the acoustic lens 14 when disposed. In the present embodiment, the friction reducing agent 14c is dispersed and added to the base material 14a, and thereby a part of the friction reducing agent 14c is exposed from a base material surface 14d on the lens surface S1.

When viewed from the outside, the friction reducing agent 14c exposed from the base material surface 14d is disposed in a state in which the friction reducing agent 14c covers the base material surface 14d. The friction reducing agent 14c exposed from the base material surface 14d constitutes the lens surface S1 along with the filler 14b exposed from the base material surface 14d and the base material surface 14d.

A material of the friction reducing agent 14c is not particularly limited as long as the material is a solid that can improve a slip characteristic of the lens surface S1 compared to a slip characteristic of the base material surface 14d.

For example, a solid lubricant that is easily subjected to shear fracture or slip deformation by an external force and thereby can improve a slip characteristic may be used as the friction reducing agent 14c.

For example, a solid lubricant formed of a laminated structure particle in which layered molecular structures are bonded by an intermolecular force may be used as the friction reducing agent 14c.

For example, a solid lubricant formed of a material having a small coefficient of friction of a surface like a fluororesin or the like may be used as the friction reducing agent 14c.

However, the friction reducing agent 14c is not limited to the solid lubricant as described above. For example, an inorganic material or an organic material that is not necessarily referred to as a solid lubricant may be used as the friction reducing agent 14c as long as the solid lubricant has a smaller coefficient of friction than the base material surface 14d.

Since FIG. 3 is a schematic view, the friction reducing agent 14c is represented in a spherical shape. However, the shape of the friction reducing agent 14c is not limited to the spherical shape. For example, the shape of the friction reducing agent 14c may be a granular shape, a polyhedral shape, a plate shape, a rod shape, a fabric shape, an indefinite shape, or the like in addition to the spherical shape.

Materials suitable for the friction reducing agent 14c include molybdenum disulfide, tungsten disulfide, graphite, graphite fluoride, boron nitride, mica, talc, calcium fluoride, silicon dioxide, fullerenes, carbon nanotubes, lead monoxide, gold, silver, tin, lead, copper, polytetrafluoroethylene (PTFB) (a fluororesin), perfluoroalkoxyfluororesin (PFA) (a fluororesin), a polyamide resin, a polyacetal resin, and so on.

The friction reducing agent 14c contained in the acoustic lens 14 is not limited to one type. For example, one or more materials selected from the group consisting of the materials listed as examples above may be used as the friction reducing agent 14c.

An exposed shape, an exposed area, and a distribution density of an exposed part of the friction reducing agent 14c on the base material surface 14d are not particularly limited as long as the coefficient of friction of the lens surface S1 can be reduced compared to the coefficient of friction of the base material surface 14d.

For example, the friction reducing agent 14c may be exposed in an adequate shape such as a granular shape, an insular shape, a layered shape, or the like on the base material surface 14d. As an example, a case where the friction reducing agent 14c is exposed in the granular shape in a range of a particle size or less is depicted in FIG. 3.

It is more preferable that the coefficient of friction on the lens surface S1 have a coefficient of dynamic friction smaller than 0.3.

It is more preferable that the particle size of the friction reducing agent 14c be smaller than or equal to 10 μm. When the particle size of the friction reducing agent 14c exceeds 10 μm, the attenuation factor of ultrasonic waves may become too large in the acoustic lens 14. When the attenuation factor of ultrasonic waves becomes too large in the acoustic lens 14, it becomes difficult for the ultrasonic waves to reach a deep part of the subject, and thus it becomes difficult to observe the deep part of the subject.

To further reduce the attenuation factor of ultrasonic waves, it is more preferable that the particle size of the friction reducing agent 14c be smaller than or equal to 6 μm.

In the present embodiment, since the friction reducing agent 14c is also dispersed to the inside of the base material 14a, the amount of the friction reducing agent 14c may also influence the acoustic characteristics of the acoustic lens 14. For this reason, the amount of the friction reducing agent 14c is set along with the amount of the filler 14b such that the acoustic characteristics required of the acoustic lens 14 are obtained.

For example, the amount of the friction reducing agent 14c in the acoustic lens 14 may be 3 parts by mass or more and 15 parts by mass or less with respect to 100 parts by mass of the base material 14a. In this case, an amount of the friction reducing agent 14c exposed to the lens surface S1 becomes appropriate, and thus a friction reducing effect caused by the friction reducing agent 14c becomes more excellent.

When the friction reducing agent 14c is less than 3 parts by mass, the amount of the friction reducing agent 14c exposed to the lens surface S1 becomes too small, and thus the coefficient of friction of the lens surface S1 may be hardly reduced.

When the friction reducing agent 14c exceeds 15 parts by mass, propagation of ultrasonic waves is obstructed by the friction reducing agent 14c, and thus it becomes easy for the attenuation factor of ultrasonic waves to increase. For this reason, a resolution of an ultrasonic image of the ultrasonic endoscope 1 may be reduced.

Each ultrasonic transducer 10 having the acoustic lens 14 according to the present embodiment is, for example, manufactured as follows.

The piezoelectric element 11 having the electrodes (not shown) provided on the respective plate surfaces 11a and 11b is joined with the acoustic matching layer 13 that is previously molded. Afterward, the substrate 50 is attached on the piezoelectric element 11 so as to extend in a surface direction. In addition, the annular members 33 and 34 are disposed at prescribed positions.

Afterward, a resin composition for forming the backing material 12 is poured into a space between the piezoelectric elements 11 and the cylindrical member 30, which space is surrounded by the annular members 33 and 34. When the resin composition is cured, the backing material 12 is formed.

Afterward, the acoustic lens 14 is disposed on a surface 13a of the acoustic matching layer 13 in a direction opposite to the piezoelectric element 11.

The acoustic lens 14 is manufactured as follows. For example, the base material 14a, the filler 14b, and the friction reducing agent 14c are mixed and thus a mixture thereof is formed. The mixture is molded in the shape of the acoustic lens 14, for example, by press working or the like and is vulcanized.

The acoustic lenses 14 manufactured in this way are bonded to the acoustic matching layer 13 each other by an adhesive whose acoustic impedance is adjusted to a value between the acoustic impedance of the acoustic matching layer 13 and the acoustic lens 14.

In this way, the ultrasonic transducer 10 is manufactured.

However, the method of forming the acoustic lens 14 is not limited to the aforementioned method. For example, the acoustic lens 14 may be formed as follows.

First, a molding die for the acoustic lens 14 is disposed around the acoustic matching layer 13. A resin composition for forming the acoustic lens 14 is poured into the molding die. Before this resin composition is cured, heat curing is performed in a state in which this resin composition is in contact with the acoustic matching layer 13 mounted on the piezoelectric element 11. Thus, the acoustic lens 14 is joined to the acoustic matching layer 13. When the acoustic lens 14 is cured, the molding die is removed.

Next, an operation of the acoustic lens 14 will be described.

Since the acoustic lens 14 contains the filler 14b in the base material 14a, the amount of the filler 14b is appropriately set, and thereby acoustic characteristics preferred as the acoustic lens of the ultrasonic endoscope is obtained.

An acoustic impedance is obtained by a density of a medium×the speed of sound in the medium. The acoustic impedance needs to be set to an appropriate value depending on a subject. For example, the filler 14b is different in density from the base material 14a, and thus the acoustic impedance is adjusted by changing the amount of the filler 14b with respect to the base material 14a. In a case where the density of the filler 14b is higher than that of the base material 14a, the acoustic impedance can be increased compared to the case of the base material 14a alone by increasing the amount of the filler 14b.

In a case where a high-density material is used as the friction reducing agent 14c, an added amount of the filler 14b is appropriately adjusted such that the acoustic lens 14 obtains necessary acoustic characteristics by combining the friction reducing agent 14c and the filler 14b.

As shown in FIG. 3, a part of the friction reducing agent 14c is exposed from the base material surface 14d in the acoustic lens 14. When a contact member G comes into contact with the lens surface S1, the friction reducing agent 14c comes into contact with a part of the contact member G. For this reason, a contact area between the base material 14a having worse slip characteristics than the friction reducing agent 14c and the contact member G is reduced. A frictional force between the contact member G and the acoustic lens 14 is reduced at a contact portion between the contact member G and the friction reducing agent 14c due to a friction reducing effect of the friction reducing agent 14c.

The friction reducing effect of the friction reducing agent 14c varies according to the material of the friction reducing agent 14c.

For example, in a case where the friction reducing agent 14c is formed of a material, such as molybdenum disulfide, which has a crystalline structure that is easy to undergo shear deformation. The friction reducing agent 14c that is in contact with the contact member G is subjected to shear deformation, and thus the contact member G becomes easy to slide.

For example, in a case where the friction reducing agent 14c is formed of a material, such as graphite, in which layered molecular structures are bonded each other by a weak intermolecular force, the layered molecular structures of the friction reducing agent 14c that is in contact with the contact member G slip with each other by the external force from the contact member G, and thus the contact member G becomes easy to slide.

For example, in a case where the friction reducing agent 14c is formed of a material, such as a fluororesin, in which the coefficient of friction of the surface is small, a frictional force itself applied to the contact member G from the friction reducing agent 14c becomes small, and thus the contact member G becomes easy to slide.

In this way, the friction reducing agent 14c is exposed to the base material surface 14d in the acoustic lens 14, and thus an actual coefficient of friction of the lens surface S1 is reduced. Thus, the contact member G becomes easy to slide on the lens surface S1.

As a result, since the contact member G is hardly caught on the base material surface 14d or the filler 14b protruding from the base material surface 14d, the base material 14a can be prevented from being broken by an external force from the contact member G.

For example, before and after use of the ultrasonic endoscope 1, the lens surface S1 of the acoustic lens 14 is cleaned with a cleaning member such as gauze. In this case, the cleaning member is the contact member G. Since the actual coefficient of friction of the lens surface S1 is reduced in the acoustic lens 14, the cleaning member is hardly caught on the lens surface S1. As a result, the acoustic lens 14 is prevented from being broken during cleaning work. In this way, durability of the acoustic lens 14 is improved, and thus a life span of the ultrasonic endoscope 1 is also prolonged.

For example, in a case where the endoscopic channel is provided in the ultrasonic endoscope 1, the endoscopic device is taken in and out from the opening that becomes the exit of the endoscopic channel in the rigid distal end portion 5. In this case, when the endoscopic device moves into the body of a patient, a metal part or a resin part of the endoscopic device may also come into contact with the lens surface S1 of the acoustic lens 14 as the contact member G. However, since the actual coefficient of friction of the lens surface S1 is low in the acoustic lens 14, the metal portion or the resin portion of the endoscopic device is also hardly caught on the lens surface S1. As a result, the acoustic lens 14 is prevented from being broken by contact with the endoscopic device. Since the durability of the acoustic lens 14 is improved in this way, the life span of the ultrasonic endoscope 1 is also prolonged.

As described above, according to the acoustic lens 14 and the ultrasonic endoscope 1 according to the present embodiment, durability against physical contact can be improved.

### Second Embodiment

Next, an ultrasonic endoscope acoustic lens according to a second embodiment will be described.

FIG. 4 is a schematic sectional view showing an example of an ultrasonic endoscope acoustic lens according to a second embodiment of the present invention.

An acoustic lens (an ultrasonic endoscope acoustic lens) 64 of the present embodiment of which main parts are shown in FIG. 4 can be used in place of the acoustic lens 14 in the ultrasonic endoscope 1 of the first embodiment (see FIG. 2). The acoustic lens 64 has the same shape as the acoustic lens 14 of the first embodiment. The acoustic lens 64 is different in internal structure from the acoustic lens 14.

Hereinafter, a description will be made focusing on a difference from the first embodiment.

The acoustic lens 64 includes a lens main body 64A and a surface layer 64B.

A filler 14b is dispersed to a base material 14a, and thus the lens main body 64A is formed. A shape (a layer thickness) of the lens main body 64A is formed in a shape (a layer thickness) that is obtained by subtracting a thickness of the surface layer 64B (to be described below) from the lens surface S1 of the acoustic lens 14 in the first embodiment.

The surface layer 64B covers a surface 64d in the lens main body 64A on the whole, and is formed in a layered shape. A surface 64a of the surface layer 64B constitutes a lens surface S2 that is a surface of the acoustic lens 64.

The surface layer 64B includes a binder resin 65, and a friction reducing agent 14c that is the same as that of the first embodiment.

The binder resin 65 holds the friction reducing agent 14c. A material of the binder resin 65 is not particularly limited if it is a resin material that can hold the friction reducing agent 14c and can fix the base material 14a by coming into close contact with the base material 14a. For example, examples of the binder resin 65 include a solvent-soluble fluororesin, an acrylic resin, an epoxy resin, a phenol resin, polyamide imide, polyimide, a silicone resin, a polyether ether ketone (PEEK) resin, PFA, and so on.

A material whose coefficient of friction is lower than that of the base material 14a is more preferably used as the material of the binder resin 65. In this case, the binder resin 65 also functions as the friction reducing agent. For example, one capable of holding the friction reducing agent 14c among the resin materials included in the examples of the friction reducing agent 14c in the first embodiment may be used as the material of the binder resin 65.

An added amount of the friction reducing agent 14c in the surface layer 64B is an adequate added amount by which a coefficient of friction on the lens surface S2 becomes lower than the coefficient of friction of the base material 14a. For example, in a slip characteristic of the lens surface S2, a coefficient of dynamic friction is more preferably less than 0.3.

For example, in a case where the same friction reducing agent 14c as in the first embodiment is used as the friction reducing agent 14c of the present embodiment, a blended amount of the friction reducing agent 14c in the present embodiment may be set such that an exposed area of the friction reducing agent 14c from the binder resin 65 on the lens surface S2 becomes similar to that from the base material 14a in the first embodiment. The blended amount of the friction reducing agent 14c in the present embodiment may be set such that a distribution density of the friction reducing agent 14c exposed from the binder resin 65 on the lens surface S2 becomes similar to that of the friction reducing agent 14c exposed from the base material 14a in the first embodiment.

For example, the amount of the friction reducing agent 14c in the acoustic lens 64 may be 3 parts by mass or more and 15 parts by mass or less with respect to 100 parts by mass of the binder resin 65. In this case, an amount of the friction reducing agent 14c exposed to the lens surface S2 becomes appropriate, and thus a friction reducing effect caused by the friction reducing agent 14c becomes more excellent.

The layer thickness of the surface layer 64B is not particularly limited if the surface layer 64B is capable of holding the friction reducing agent 14c exposed to the lens surface S2. For example, the layer thickness of the surface layer 64B may be 100% or more and 300% or less of a maximum particle size of the friction reducing agent 14c.

The acoustic lens 64 having this constitution is manufactured as follows. For example, the base material 14a and the filler 14b are mixed and thus a mixture thereof is formed. The mixture is molded in the shape of the acoustic lens 64, for example, by press working or the like and is vulcanized. Afterward, a coating liquid in which the binder resin 65, a solvent, and the friction reducing agent 14c are mixed is coated on a surface of the lens main body 64A.

Afterward, adequate drying treatment of volatilizing the solvent of the coating liquid is performed. Thus, the surface layer 64B is formed on the surface 64d of the lens main body 64A, and the acoustic lens 64 is manufactured.

The acoustic lens 64 manufactured in this way is joined to the acoustic matching layer 13 in the same way as in the first embodiment. Thus, the ultrasonic transducer 10 of the present embodiment is manufactured.

Next, an operation of the acoustic lens 64 will be described.

The acoustic lens 64 contains the filler 14b in the base material 14a in the lens main body 64A. For this reason, the amount of the filler 14b is appropriately set, and thereby acoustic characteristics preferred as the acoustic lens of the ultrasonic endoscope are obtained in the same way as in the first embodiment.

Since the friction reducing agent 14c is not contained in the lens main body 64A in the present embodiment, the acoustic characteristics of the acoustic lens 64 are substantially determined by the base material 14a and the filler 14b.

The friction reducing agent 14c is dispersed only around the surface layer 64B of the acoustic lens 64 in the present embodiment. For this reason, an added amount of the friction reducing agent 14c which the lens surface S2 requires to have the same frictional characteristics as in the first embodiment is remarkably reduced. Therefore, to curb an influence on the acoustic characteristics of the acoustic lens 64 of the friction reducing agent 14c, a need to reduce the added amount of the friction reducing agent 14c or accurately adjust the added amount of the friction reducing agent 14c is remarkably reduced.

For example, in the case of the first embodiment, when the friction reducing agent 14c is distributed unevenly in the base material 14a, a variation in the acoustic characteristics of the acoustic lens 14 is easy to occur. For this reason, there is a need to select a material having a good distribution characteristic in the base material 14a as the friction reducing agent 14c.

However, in the present embodiment, corresponding to the material of the friction reducing agent 14c, the binder resin 65 by which the distribution characteristic is improved is selected, and thereby a variation of the friction reducing agent 14c can be easily curbed. To begin with, in the present embodiment, the added amount itself of the friction reducing agent 14c does not contribute much to the acoustic characteristics of the acoustic lens 64. For this reason, even if there is a variation in the distribution of the friction reducing agent 14c, an influence on the acoustic characteristics of the acoustic lens 64 is small. With regard to the slip characteristics, if a coefficient of friction lower than a constant coefficient of friction is obtained, slip characteristics resistant to breakage are obtained. For this reason, an allowable range is wide with regard to a distribution variation in a direction in which the distribution of the friction reducing agent 14c becomes dense.

According to the present embodiment, a selection range of the material of the friction reducing agent 14c becomes wide.

According to the acoustic lens 64, in the same way as in the first embodiment, an actual coefficient of friction of the lens surface S2 can be reduced depending on an exposed amount of the friction reducing agent 14c on the lens surface S2. For this reason, according to the present embodiment, a contact member G (not shown) becomes easy to slide on the lens surface S2.

As a result, the contact member G is hardly caught on the lens surface S2, and thus the surface layer 64B and the lens main body 64A can be prevented from being broken by an external force from the contact member G. In this way, durability of the acoustic lens 64 is improved.

In particularly, in the present embodiment, the material having a lower coefficient of friction than the base material 14a is selected as the material of the binder resin 65, and thus the actual coefficient of friction of the lens surface S2 can be further reduced.

As in the present embodiment, in a case where the entire lens surface S2 is covered by the surface layer 64B, the durability of the acoustic lens 64 is further improved in that the surface layer 64B has a protective function of preventing direct contact between the contact member G and the base material 14a.

In the present embodiment, the entire lens main body 64A is covered by the surface layer 64B, and thus the filler 14b in the lens main body 64A is not exposed to the lens surface S2. For this reason, the slip characteristics are prevented from being deteriorated by the exposure of the filler 14b.

As described above, according to the acoustic lens 64 of the present embodiment, durability against physical contact can be improved.

### Third Embodiment

Next, an ultrasonic endoscope acoustic lens of a third embodiment will be described.

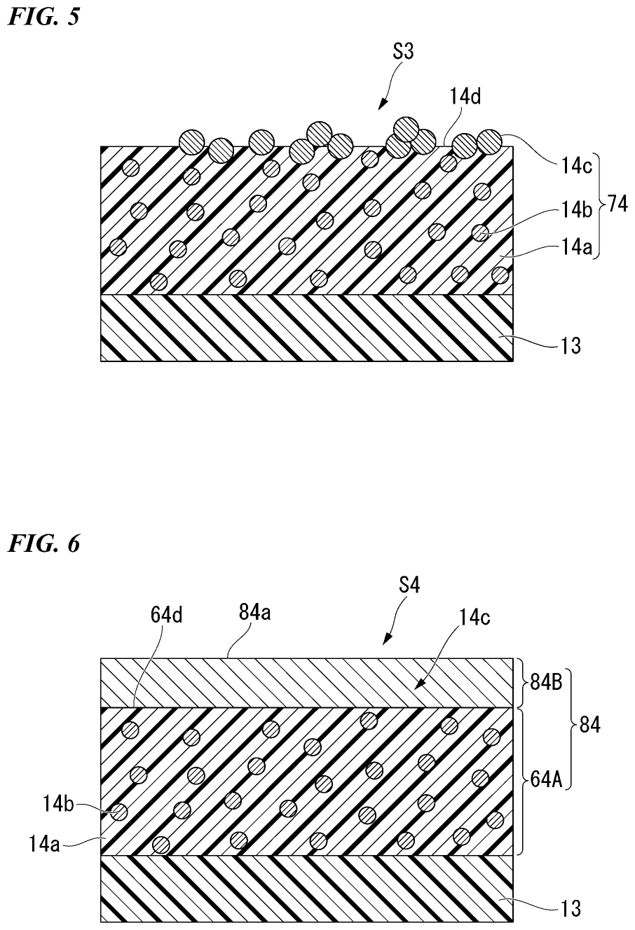

FIG. 5 is a schematic sectional view showing an example of an ultrasonic endoscope acoustic lens according to a third embodiment of the present invention.

An acoustic lens (an ultrasonic endoscope acoustic lens) 74 of the present embodiment of which main parts are shown in FIG. 5 can be used in place of the acoustic lens 14 in the ultrasonic endoscope 1 of the first embodiment (see FIG. 2). The acoustic lens 74 has the same shape as the acoustic lens 14 of the first embodiment. The acoustic lens 74 is different in internal structure from the acoustic lens 14.

Hereinafter, a description will be made focusing on a difference from the first embodiment.

Like the first embodiment, the acoustic lens 74 includes a base material 14a, a filler 14b, and a friction reducing agent 14c. However, in the present embodiment, the friction reducing agent 14c is disposed only around a base material surface 14d. For this reason, like the lens surface S1 in the first embodiment, a lens surface S3 of the acoustic lens 74 has the filler 14b and the friction reducing agent 14c exposed from the base material surface 14d. However, in the present embodiment, since the friction reducing agent 14c is distributed only around the base material surface 14d, an added amount of the friction reducing agent 14c is remarkably small compared to the first embodiment.

Like the first embodiment, the friction reducing agent 14c in the present embodiment is disposed to cover a part of the base material surface 14d. The friction reducing agent 14c in the present embodiment may be exposed in an adequate shape such as a granular shape, an insular shape, or the like. An example in which the friction reducing agent 14c is exposed in an insular shape that is larger than a particle size of an individual particle is depicted as an example in FIG. 5.

Like the first embodiment, an exposed shape, an exposed area (an exposed amount), and a distribution density of an exposed part of the friction reducing agent 14c on the lens surface S3 are appropriately set such that a coefficient of friction of the lens surface S3 becomes lower than that of the base material 14a. For example, it is more preferable that the coefficient of friction on the lens surface S3 be smaller than 0.3 as a coefficient of dynamic friction.

The acoustic lens 74 having this constitution is manufactured as follows. For example, the base material 14a and the filler 14b are mixed and thus a mixture thereof is formed. The mixture is molded in the shape of the acoustic lens 74, for example, by press working or the like and is vulcanized. Afterward, the friction reducing agent 14c is deposited on a surface of the molding. The method of depositing the friction reducing agent 14c is not particularly limited if fixing strength by which the friction reducing agent 14c is hardly peeled off by contact with a contact member G (not shown) is obtained. For example, the method of depositing the friction reducing agent 14c includes a sputtering method, electroless plating, a rubbing method, a tumbling method, an impingement method, an ion plating method, a thermal chemical vapor deposition (CVD), a plasma CVD, or the like. For example, a physical vapor deposition (PVD) or CVD in addition to those provided as exemplary examples above may be used as the method of depositing the friction reducing agent 14c.

For example, in a case where the friction reducing agent 14c has a strong adsorption force against the base material 14a, a powder of the friction reducing agent 14c may be only dusted on the base material surface 14d.

In this way, when the deposition of the friction reducing agent 14c on a surface of the base material surface 14d is completed, the acoustic lens 74 is manufactured.

The acoustic lens 74 manufactured in this way is joined to an acoustic matching layer 13 in the same way as in the first embodiment. Thus, an ultrasonic transducer 10 of the present embodiment is manufactured.

Next, an operation of the acoustic lens 74 will be described.

Like the second embodiment, the friction reducing agent 14c of the acoustic lens 74 is disposed on the lens surface S3 and only therearound. In this respect, the acoustic lens 74 has the same operation as the acoustic lens 64 of the second embodiment.

However, in the present embodiment, the friction reducing agent 14c is directly deposited around the base material surface 14d without a binder resin 65. For this reason, the friction reducing agent 14c is fixed by a fixing force between the friction reducing agent 14c and the base material 14a.

In this way, the acoustic lens 74 does not have a layered structure such as the base material 14a and the binder resin 65 in the second embodiment. For this reason, a variation or the like in a focusing characteristic of ultrasonic waves caused by a layer thickness variation or the like of the binder resin 65 may not occur. Further, since stress caused by a difference in thermal expansion coefficient or the like between the base material 14a and the binder resin 65 does not occur, durability against sterilization treatment or the like is improved.

As described above, according to the acoustic lens 74 of the present embodiment, durability against physical contact can be improved.

### Fourth Embodiment

Next, an ultrasonic endoscope acoustic lens of a fourth embodiment will be described.

FIG. 6 is a schematic sectional view showing an example of an ultrasonic endoscope acoustic lens according to a fourth embodiment of the present invention.

An acoustic lens (an ultrasonic endoscope acoustic lens) 84 of the present embodiment of which main parts are shown in FIG. 6 can be used in place of the acoustic lens 14 in the ultrasonic endoscope 1 of the first embodiment (see FIG. 2). The acoustic lens 84 has the same shape as the acoustic lens 14 of the first embodiment.

The acoustic lens 84 includes a surface layer 84B in place of the surface layer 64B in the second embodiment.

Hereinafter, description will be made focusing on a difference from the second embodiment.

Like the surface layer 64B in the second embodiment, the surface layer 84B is formed in a layered shape that covers a surface 64d in a lens main body 64A on the whole. A surface 84a of the surface layer 84B constitutes a lens surface S4 that is a surface of the acoustic lens 84.

A friction reducing agent 14c that is similar to that of the first embodiment is deposited in a layered shape, and the surface layer 84B is formed. However, since the friction reducing agent 14c is densely deposited, a granular shape is not shown in FIG. 6. Since FIG. 6 is a schematic view, it is represented that a layer thickness of the surface layer 84B is constant. However, the layer thickness of the surface layer 84B may be set to an adequate value that is greater than or equal to a particle size of the friction reducing agent 14c if an influence on acoustic characteristics of the acoustic lens 84 is an allowable range. The layer thickness of the surface layer 84B may be changed according to a place if an influence on the acoustic characteristics of the acoustic lens 84 is an allowable range. The surface 84a in the surface layer 84B may have a fine uneven shape if a necessary coefficient of friction is obtained.

In the acoustic lens 84 having this constitution, in the same way as in the second embodiment, after the lens main body 64A is formed, the friction reducing agent 14c is deposited on the surface 64d in the lens main body 64A in a layered shape by covering the entire surface 64d. The same method of depositing the friction reducing agent 14c as in the third embodiment may be used as the method of depositing the friction reducing agent 14c.

In this way, when the formation of the surface layer 84B is completed on the surface 64d, the acoustic lens 84 is manufactured.

The acoustic lens 84 manufactured in this way is joined to an acoustic matching layer 13 in the same way as in the first embodiment. Thus, an ultrasonic transducer 10 of the present embodiment is manufactured.

Next, an operation of the acoustic lens 84 will be described.

Like the second embodiment, the friction reducing agent 14c of the acoustic lens 84 is disposed only on the lens surface S4 and therearound. In this respect, the acoustic lens 84 has the same operation as the acoustic lens 64 of the second embodiment.

Further, in the present embodiment, as in the third embodiment, the friction reducing agent 14c is directly deposited over the entire lens surface S4. In other words, the friction reducing agent 14c is directly deposited on the surface 64d of the lens main body 64A without a binder resin 65. In this respect, the acoustic lens 84 also has the same effect as in the third embodiment. Like the present embodiment, in a case where the entire lens surface S4 is covered by the friction reducing agent 14c, durability of the acoustic lens 84 is further improved in that the surface layer 84B has a protective function of preventing direct contact between a contact member G and a base material 14a.

As described above, according to the acoustic lens 84 of the present embodiment, durability against physical contact can be improved.

In the description of each embodiment, the case where the ultrasonic endoscope acoustic lens is used in the ultrasonic endoscope has been described by way of example. However, the ultrasonic endoscope acoustic lens may be used in various medical instruments for measuring ultrasonic waves or instruments other than the medical instruments.

### EXAMPLES

Hereinafter, Examples 1 to 4 of the ultrasonic endoscope acoustic lens of each embodiment will be described along with Comparative Example.

Constitutions and evaluation results of the ultrasonic endoscope acoustic lenses of Examples 1 to 4 and Comparative Example are shown in Table 1 below. However, the reference signs of member names are omitted in Table 1.

TABLE 1

| | | | | | | | | Evaluation results | | |
|---|---|---|---|---|---|---|---|---|---|---|
| | Base material | | Filler | | Friction reducing agent | | | | Acoustic | |
| | | Parts | | Parts | | Parts | | Coefficient | IMP | Compre- |
| | Material | by mass | Material | by mass | Material | by mass | Distributed state | of friction | (Pa · s/m³) | hensive |
| Example 1 | Polysiloxane | 100 | Silica | 30 | MoS₂ | 5 | Dispersed in | 0.28 | 1.38 | A |
| | | | | | | | base material | | | |
| Example 2 | Polysiloxane | 100 | Silica | 30 | PTFE | 5 | Distributed on surface | 0.29 | 1.3 | A |
| | | | | | particle | | of lens main body | | | |
| | | | | | | | in layered shape | | | |
| Example 3 | Polysiloxane | 100 | Silica | 30 | PTFE | 5 | Distributed on surface | 0.27 | 1.33 | A |
| | | | | | particle | | of lens main body | | | |
| | | | | | | | in layered shape | | | |
| Example 4 | Polysiloxane | 100 | Silica | 30 | Graphite | 5 | Dispersed in | 0.28 | 1.39 | A |
| | | | | | | | base material | | | |
| Comparative | Polysiloxane | 100 | Silica | 30 | — | — | — | 0.36 | 1.35 | B |
| Example | | | | | | | | | | |

### Example 1

Example 1 is an example that relates to the acoustic lens 14 of the first embodiment. However, evaluation was performed by a sheet-like test sample (equally applied to each of Examples and Comparative Example below).

As shown in Table 1, a silicone rubber compound containing dimethylpolysiloxane, which is a silicone rubber compound (written as “polysiloxane” in Table 1) using a diorganopolysiloxane as a main agent, in a main skeleton was used as the base material 14a of Example 1.

Silica having an average particle size of 3 μm was used as the filler 14b. Here, the average particle size was measured by a laser diffraction method (equally applied to the following average particle size). The filler 14b was added at a fraction of 30 parts by mass based on 100 parts by mass of the cured base material 14a.

Molybdenum disulfide (MoS₂) having an average particle size of 5 μm was used as the friction reducing agent 14c. The friction reducing agent 14c was added at a fraction of 5 parts by mass based on 100 parts by mass of the cured base material 14a.

The base material 14a, the filler 14b, and the friction reducing agent 14c were mixed at a blending ratio as described above, and were injection-molded using a molding die, and thereby a test sample of Example 1 was manufactured. The cured test sample was a sheet having an external shape of 100 mm×50 mm×0.5 mm.

### Example 2

Example 2 is an example that relates to the acoustic lens 64 of the second embodiment.

Materials and added amounts of the base material 14a and the filler 14b of Example 2 were the same as those of Example 1.

A solvent-soluble fluororesin containing fluoroethylene vinyl ether (FEVE) as a main agent was used as the binder resin 65 of the surface layer 64B. A PTFE powder having an average particle size of 5 μm (however, a maximum particle size was 10 μm or less) was used as the friction reducing agent 14c of the surface layer 64B. The friction reducing agent 14c was added at a fraction of 5 parts by mass when the dried binder resin 65 was set to 100 parts by mass.

In this example, the binder resin 65 is also a fluororesin, and functions as the friction reducing agent. For this reason, this example becomes an example of a case where a plurality of types of friction reducing agents are contained in the acoustic lens 64.

In a test sample of Example 2, the base material 14a and the filler 14b were mixed at a blending ratio as described above, and were injection-molded using a molding die, and thereby a sheet body corresponding to the lens main body 64A was manufactured.

The friction reducing agent 14c was dispersed in the binder resin 65 dissolved in a solution, and thereby a coating liquid was produced. The coating liquid was uniformly spray-coated on a surface of the sheet body. The sheet body coated with the coating liquid was dried by heating at a temperature of 120° C. Thus, a solvent of the coating liquid was volatilized, and solid components of the friction reducing agent 14c and the binder resin 65 were deposited on the surface of the sheet body in a layered shape, so that the surface layer 64B was formed. A layer thickness of the surface layer 64B was less than or equal to 10 μm. A shape of the cured sheet body was set to 100 mm×50 mm×0.5 mm.

### Example 3

Example 3 is an example that relates to the acoustic lens 74 of the third embodiment.

Materials and added amounts of the base material 14a and the filler 14b of Example 3 were the same as those of Example 1.

A PTFE powder having an average particle size of 5 μm (however, a maximum particle size was 10 μm or less) was used as the friction reducing agent 14c. The friction reducing agent 14c was used by 5 parts by mass based on 100 parts by mass of the cured base material 14a.

After the same sheet body as in Example 2 was manufactured at a blending ratio as described above, the friction reducing agent 14c was deposited on a surface of the sheet body by a sputtering method, and thereby a test sample of Example 3 was manufactured. In the test sample of Example 3, the surface of the sheet body was coated within a range of about 5% with the friction reducing agent 14c distributed in an insular shape in a top view.

### Example 4

Example 4 is an example that relates to the acoustic lens 14 of the first embodiment.

In Example 4, in place of MoS₂ of Example 1, graphite was used as the friction reducing agent 14c. The graphite was added at a fraction of 5 parts by mass based on 100 parts by mass the cured base material 14a.

### Comparative Example

Silica having an average particle size of 3 μm was added as a filler using a polysiloxane equal to that of Example 1 as a base material, and thereby a test sample of Comparative Example was manufactured. The silica was added at a fraction of 30 parts by mass based on 100 parts by mass of the cured base material. The test sample of Comparative Example became a sheet body having the same external shape as in Example 1.

No friction reducing agent was added to the test sample of Comparative Example.

[Evaluation Method]

As shown in Table 1, as evaluation of the test samples, coefficient of friction evaluation, acoustic characteristic evaluation, and comprehensive evaluation were performed.

In the coefficient of friction evaluation, a coefficient of dynamic friction of each test sample was measured according to JIS K7129:1999. However, mass of slip piece was 100 g, and a speed of the slip piece was 500 mm/min SUS304 was used as a material of a counterpart member of each test sample.

It was determined that the coefficient of dynamic friction was good in the case of 0.3 or less, and poor in the case of 0.3 or more.

In the acoustic characteristic evaluation, an acoustic impedance was measured. The acoustic impedance was an amount that related to an image resolution.

A method according to a water immersion multiple reflection method without using a contrast measurement piece in the method for measurement of ultrasonic attenuation coefficient of solids (JIS Z 2354) was used as a method for measuring an acoustic impedance (described in Table 1 as “acoustic IMP”). In this case, the ultrasonic transducer for measurement was driven at a frequency of 5 MHz.

It was determined that the acoustic impedance was good in the case of 1.2 Pa·s/m³ or more and 1.4 Pa·s/m³, and poor in the case of being less than 1.2 or exceeding 1.4.

In the comprehensive evaluation, it was determined that each test sample was “good” (“A” in Table 1) when the coefficient of dynamic friction and the acoustic impedance were good, and “no good” (“B” in Table 1) when at least one of the coefficient of dynamic friction and the acoustic impedance was poor.

[Evaluation Results]

As shown in Table 1, since the coefficient of dynamic frictions of Examples 1 to 4 were 0.28, 0.29, 0.27, and 0.28, all of Examples 1 to 4 were determined to be good. In Examples 1 to 4, it was considered that the coefficient of dynamic friction was reduced due to an effect of the friction reducing agent.

In contrast, in Comparative Example, it was determined that the test sample was no good because the coefficient of dynamic friction was 0.36.

It was understood that, in Comparative Example, the silica was exposed from the base material, but even if the silica was exposed, an effect of reducing the coefficient of dynamic friction was not obtained. For this reason, the silica did not function as the friction reducing agent.

Since the acoustic impedances of Examples 1 to 4 and Comparative Example were 1.38 Pa·s/m³, 1.30 Pa·s/m³, 1.33 Pa·s/m³, 1.39 Pa·s/m³, and 1.35 Pa·s/m³, all of Examples 1 to 4 and Comparative Example were determined to be good. This was considered to be because the same amount of silica was used as the filler in common with Examples and Comparative Example, and the acoustic characteristics were decided by the added amount of the silica.

As the comprehensive evaluation, it was determined that Examples 1 to 4 were good, and Comparative Example was no good.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. An ultrasonic endoscope acoustic lens comprising:
a base material composed of at least one elastomer;
a filler added to the base material; and
a friction reducing agent disposed to cover at least a part of a surface of the base material and exposed to a lens surface.

2. The ultrasonic endoscope acoustic lens according to claim 1,
wherein the friction reducing agent contains one or more materials selected from a group consisting of a fluororesin, molybdenum disulfide, graphite, boron nitride, a polyamide resin, a polyacetal resin, and tungsten disulfide.

3. The ultrasonic endoscope acoustic lens according to claim 1,
wherein a particle size of the friction reducing agent is less than or equal to 10 μm.

4. The ultrasonic endoscope acoustic lens according to claim 1,
wherein an amount of the friction reducing agent is 3 parts by mass or more and 15 parts by mass or less based on 100 parts by mass of the base material.

5. The ultrasonic endoscope acoustic lens according to claim 1,
wherein a coefficient of dynamic friction on the lens surface is smaller than 0.3.

6. The ultrasonic endoscope acoustic lens according to claim 1,
wherein the base material is composed of a diorganopolysiloxane or a silicone rubber compound containing the diorganopolysiloxane as a main agent.

7. The ultrasonic endoscope acoustic lens according to claim 1,
wherein the friction reducing agent is disposed on the surface of the base material in a layered shape.

8. The ultrasonic endoscope acoustic lens according to claim 1,
wherein the friction reducing agent is fixed to the surface of the base material.

9. An ultrasonic endoscope comprising the ultrasonic endoscope acoustic lens according to claim 1.
